(19) United States
(12) Patent Application Publication
(10) Pub. No.: US 2012/0225992 A1
(43) Pub. Date: Sep. 6, 2012

(54) AMINO AND HYDROXYL FUNCTIONAL COMPOUNDS

(75) Inventors: Mohamad Deeb SHALATI, LOUISVILLE, KY (US); NANDA Ajaya, LOUISVILLE, KY (US); HELLEMS Stephen, LOUISVILLE, KY (US)

(73) Assignee: NUPLEX RESINS B.V., BERGEN OP ZOOM (NL)

(21) Appl. No.: 13/472,582

(22) Filed: May 16, 2012

Related U.S. Application Data
(63) Continuation of application No. PCT/EP2010/067714, filed on Nov. 17, 2010.
(60) Provisional application No. 61/261,779, filed on Nov. 17, 2009.

(30) Foreign Application Priority Data
Nov. 27, 2009 (EP) 09177390.3

(51) Int. Cl. C07C 229/30; C08G 18/68; C09D 175/00; C08F 267/06

(52) U.S. Cl. 524/507; 560/170; 525/285; 528/75; 524/874

(57) ABSTRACT

The invention relates to amino and hydroxy-functional compounds other than polyesters, wherein the compound includes an amine in the form of aspartic acid esters functionality, and wherein the amino and hydroxy-functional compound has (a) a molecular weight (Mn) of at least about 500, (b) an acid value of about 5 or less, (c) a hydroxyl value of about 30 or more, and (d) an amine value of about 30 or more. Preferably, the compound includes molecules having on the average: at least 1 secondary amino group as an aspartate, and/or at least 1 hydroxy group, and an average total functionality of about 1.8 or higher.

### CROSS REFERENCE TO RELATED APPLICATIONS

This application is a continuation of PCT application number PCT/EP2010/067714 filed on 17 Nov. 2010, which claims priority from U.S. provisional application No. 61/261,779 filed on 17 Nov. 2009, and from EP application number 09177390.3, filed on 27 Nov. 2009. All applications are hereby incorporated by reference in their entireties.

### BACKGROUND OF THE INVENTION

1. Field of the Invention

The present invention relates to amino and hydroxy-functional compounds, to compositions comprising such compounds; their use as binders or as modifiers in binder systems. These binders or binder systems are particularly suitable for crosslinking with polyisocyanate compounds to give crosslinked poly(urea/urethane) systems, which are useful as coatings, adhesives, sealants or caulking materials; either as one component composition, but preferably as two-component poly(urea/urethane) hybrid coating compositions. The present invention furthermore relates to said crosslinked systems and processes for manufacturing of the amino polyols.

2. Description of the Related Art

High performance, durable coatings based on acrylic and/or polyester polyols and isocyanates are well known in the automotive and industrial coating markets. However, the increased demands for lower VOC necessitate the need to lower the molecular weight of the polyols and/or the use of low MW reactive diluents in order to increase solid contents of the coatings. As a result of incorporating these low MW polyols, the speed of drying and early hardness development of coatings has suffered, in comparison with the conventional medium or low solids one- or two-component urethane coatings. The use of hindered secondary amine compounds along with isocyanate hardeners has alleviated, to some extent, the early cure problem due to the fast amine-NCO reaction and the generation of harder and more polar urea groups in the resulting polyurea coatings.

Polyaspartate esters have been described in U.S. Pat. No. 5,126,170 and U.S. Pat. No. 5,236,741, which are hereby incorporated by reference in their entireties, for the preparation of a polyurea coating by coating the substrate with a coating composition containing a polyisocyanate component and an isocyanate-reactive component containing at least one polyaspartic acid ester and curing the composition to a temperature of 100° C. or less.

EP 1,038,897 A2, which is hereby incorporated by reference in its entirety, discloses the preparation of polyurea coatings comprising a polyisocyanate component, an isocyanate-reactive component, for coating the substrate with coating composition and curing at temperature less than 100° C. The isocyanate-reactive component comprises the reaction product of a diester of maleic or fumaric acid and polyamine; the polyamine having 2 primary amine groups and at least one other functional group which is reactive towards isocyanate at temperature below 100° C.

U.S. Pat. No. 5,596,044, which is hereby incorporated by reference in its entirety, discloses prepolymers derived from aminoalcohols, containing hydantoin group precursors and their use in coatings compositions.

U.S. Pat. No. 7,166,748, which is hereby incorporated by reference in its entirety, discloses a coating composition comprising a polyisocyanate, an isocyanate-reactive component having hydroxyl and aspartate functionality, coating a substrate with coatings composition containing amine and/or hydroxylamine compounds.

U.S. Pat. No. 5,633,389, which is hereby incorporated by reference in its entirety, discloses a thermo-reversible process for the preparation of a hydantoin comprising the reaction of unsaturated polyester with mono-functional amine to yield poly(aspartate ester), reacting the aspartate ester with isocyanate to produce a poly(ester urea) and heating the poly(ester urea) to form a hydantoin compound.

U.S. Pat. No. 5,561,214, which is hereby incorporated by reference in its entirety, describes the composition and process for making hyperbranched polyaspartate esters.

U.S. Patent Publication Application 2005/0059792 A1, which is hereby incorporated by reference in its entirety, describes a method for preparing flexible polyaspartate esters by the incorporation of unsaturated oligoester prepared by the transesterification of α,β-unsaturated esters with hydroxyl-functional compounds and reacting the transesterified product with primary di-amine and reacting the remaining primary amine groups with α,β-unsaturated esters.

Although the prior art uses aspartate esters in the manufacture of coatings, a further improvement in properties with respect to flexibility, adhesion, durability, combined with balanced cure speed remains a challenge.

### SUMMARY OF THE INVENTION

The present invention relates to amino and hydroxy-functional compounds, not being polyesters, wherein the amino functionality is a secondary amine in the form of aspartic acid esters, and wherein the amino and hydroxy-functional compounds have
(a) a molecular weight (Mn) of at least about 500
(b) an acid value of about 5 or less
(c) a hydroxyl value of about 30 or more
(d) an amine value of about 30 or more.

Preferably, the amino and hydroxy-functional molecules of the compound, not being polyesters, have on average
(e) at least 1 secondary amine group as an aspartate ester,
(f) and/or at least 1 hydroxyl group,
(g) an average total functionality of secondary amine and hydroxyl group of 1.8 or more per molecule.

The invention furthermore relates to curable compositions comprising said amino and hydroxy-functional compounds and a polyisocyanate, wherein the amount of isocyanate is present in about 60% of the molar amount of the amine and alcohol groups, or more.

In a preferred embodiment, the amino and hydroxy-functional compounds comprise either one or a mixture of the following compounds:

### Optionally, 1) Amino and Hydroxy-functional Polyester (Formula I);

### Either 2) Amino and Hydroxy-functional Acrylic (Formula II);

Or 3) Amino and Hydroxy-functional compound (Formula III).

Another preferred embodiment of the present invention comprises a blend of any or a mixture of the above amino and hydroxy-functional compounds (2) or (3) with any of the following commercially available components:

1) Hydroxy-functional polyesters
2) Hydroxy-functional acrylic polymers
3) Other hydroxy-functional polymers
4) Polyaspartic acid esters.

An amino and hydroxy-functional polyester can be used in combination with either amino and hydroxy-functional compounds (2) or (3), which polyester has the general structure according to Formula I:

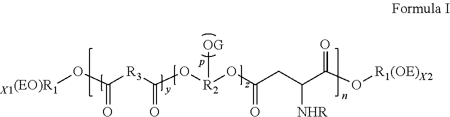

### Wherein:

R=mono-valent alkyl, aryl and/or arylalkyl radical
R₁=residue obtained from a polyol after removing the OH groups and having a valency of 1 to 6
R₂=residue obtained from a polyol after removing the OH groups and having a valency of 2 to 6
R₃=divalent saturated and/or unsaturated alkyl and/or aryl radical
E=H or acyl group having 1 to 18 carbon atoms
X¹, X²=an integer having an equal or different values of 0 to 5, but and the sum of X¹ and X² is at least 1
y, z=an integer having a value of 0 or 1
p=an integer having a value between 0 to 4
G=E and/or is a residue having the following structure:

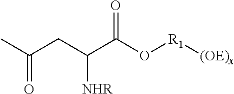

n=an integer having a value between 1 to 10
Preferably, the amino and hydroxy functional acrylic polymer has the general structure according to Formula II:

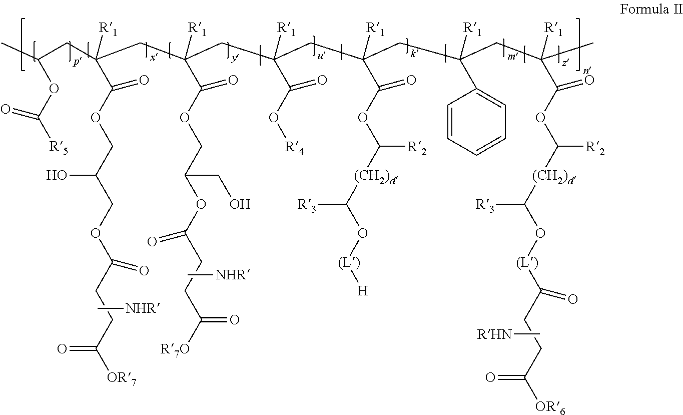

### Wherein:

R′=identical to R in Formula I
R′₁=═H, methyl
R′₂, R′₃=both H or either one is methyl and the other is H
d′=an integer having a value of 0 to 2
R′₄, R′₅=alkyl having 1 to 18 carbon atoms
R′₆,=methyl, a mixture of organic residues obtained from the addition of an epoxy compound to an acid and having the following structures:

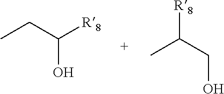

R′₈=H, alkyl, or methyl versatate radical
L′=represents nil or divalent organic residue having the following structure:

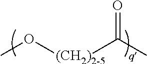

q′ is an integer having a value of 0 to 3.
z′=an integer or fraction having a value between 0 and 1
x′, y′=a fraction having a value between 0 and 0.8, the sum of x+y is an integer of 0 or 1, and the sum of x′, y′ and z′ is 1.
u′, n′, m′, p′=each is an integer having a value, independently, of 0 or 1, and the sum of
u′, n′, m′, and p′ is at least 2 and not more than 40
R′₇=monovalent alkyl radical having 1 to 18 carbon atoms

In another embodiment of the present invention, the composition comprises amino and hydroxy-functional compounds having the general structure of Formula III:

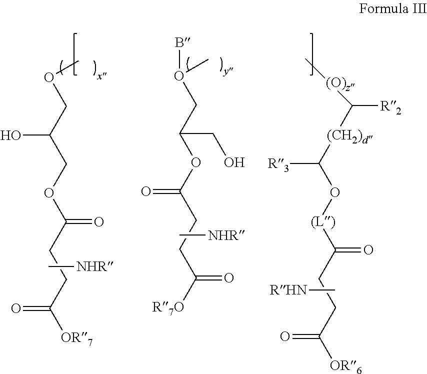

Wherein: B″ represents the backbone of Formula III and it is an organic residue obtained from either a polyol after removing the hydroxyl groups and/or from an epoxy compound after removing the oxirane rings where such a backbone is based on a hydrocarbon residue, a polyether residue, a polyurethane residue, a polycarbonate residue or any other backbone residue having a valency between 1 to 10.
x″, y″, z″, d″, L″, R″, R″₂, R″₃, R″₆, and R″₇ have the identical definitions as those for x′, y′, z′, d′, L′, R′, R′₂, R′₃, R′₆, and R′₇ described for Formula II.

The novel amino and hydroxy-functional compounds of the present invention appear to be very suitable as binders for crosslinking with polyisocyanates to give fast curing poly(urea/urethane) hybrid coatings having good flexibility, durability, chemical resistance and low VOC. None of the prior art teaches the preparation of acrylic, polyester or other polymers or oligomers having both aspartate and hydroxyl functional groups which are useful for the preparation of poly(urea/urethane) hybrid coatings in a coatings composition comprising aspartate and hydroxyl functional acrylic and/or the polyester polymers with polyisocyanate hardeners.

### BRIEF DESCRIPTION OF THE DRAWINGS

The features and advantages of the invention will be appreciated upon reference to the following drawings, in which:

FIG. 1 shows the 20° Gloss Retention of White paints based on Amino and Hydroxy-Functional Compounds at various QUV 313 exposure time according to the invention and as described in several of the examples.

### DESCRIPTION OF ILLUSTRATIVE EMBODIMENTS

The following is a description of certain embodiments of the invention, given by way of example only and with reference to the drawings.

The present invention relates to amino and hydroxy-functional compounds, not being polyesters, wherein the amine is a secondary amine in the form of aspartic acid esters, and wherein the amino and hydroxy-functional compounds have (a) a molecular weight (Mn) of at least about 500, (b) an acid value of about 5 or less, (c) a hydroxyl value of about 30 or more, and (d) an amine value of about 30 or more.

Preferably, the amino and hydroxy-functional molecules have, on average, at least about 1 secondary amine group in the form of aspartic acid esters and at least about 1 hydroxyl group, and the average total amino and hydroxy functionality is at least about 1.8 or more per molecule.

In one preferred embodiment of the invention, the amino and hydroxy-functional compound is based on a polyol having polyester or oligoester backbone, hereinafter referred to as a polymer is used in admixture with the amino and hydroxy-functional compounds that are not based on a polyester backbone.

In one preferred embodiment of the invention, the amino and hydroxy-functional compound is based on a polyol having an acrylic backbone. The polyacrylic compound may be an oligomer or polymer, which is together hereinafter referred to as a polymer.

In another preferred embodiment of the invention, the amino and hydroxy-functional compound is derived from a polyol and/or epoxy having a hydrocarbon backbone, polyether, polyurethane, polycarbonate, polyacetal, polyamide, polyimide, polythioether, polythioester, polysiloxane, or any other non-acrylic or non-ester backbone. The non-acrylic or non-ester based polyol and/or epoxy may be a monomeric, an oligomeric or a polymeric compound, which is together hereinafter referred to as a polymer.

The number average molecular weight of the amino and hydroxy-functional compound is about 500 or higher, preferably about 600 or higher, Generally, the number average molecular weight (Mn) is about 5,000 or lower, preferably about 3,000 or lower and even more preferred about 1200 or lower. The number average molecular weight may be for example in the range of 500-3000 or 600 to 5000. The molecular weight is expressed in Dalton, and the molecular weight and polydispersity are measured by GPC against polystyrene standard in THF; with a column adapted for measuring the appropriate molecular weight.

The polydispersity of the amino and hydroxy-functional compound preferably is about 4 or lower, more preferably about 2.5 or lower. Generally, the polydispersity will be about 1.2 or higher. A lower polydispersity has the advantage of improving the viscosity such that less organic solvent is required.

The acid value of the amino and hydroxy-functional compound is generally about 5 or less, and even more preferably about 3 or less, like for example between 0 and 3.

The amino and hydroxy-functional compound generally has a hydroxyl value of about 30 or higher, preferably of about 40 or higher. Generally, the hydroxyl value will be about 300 or lower, preferably about 200 or lower, and most preferable about 150 or lower. A high hydroxyl value may cause brittleness because of a too high crosslinking density. The hydroxyl value of about 30 or higher is important to achieve a stable coating with good properties. It will be appreciated that the amine value is expressed in the weight of KOH in milligrams that is equal in basicity to NH present in 1 gram compound. The hydroxyl value is also expressed in number of milligrams of KOH that will neutralize the acetic acid liberated from the reaction of an OH-functional compound with acetic acid anhydride.

The amino and hydroxy-functional compound generally has an amine value of about 30 or higher, preferably an amine value of about 40 or higher. Generally, the amine value will be about 300 or lower, preferably about 200 or lower. Most preferred is an amine value between 50 and 150. The amino-functionality is an aspartate-type functionality.

The amino and hydroxy-functional compound preferably has an average functionality of about 1.8 or more, preferably about 2.0 or more, and more preferable about 2.5 or more, and even more preferably about 3 or more. Generally, the average functionality will be about 10 or less, preferably about 5 or less. The functionality may be for example in the range between 2 and 5, or 2 and 10.

The hydroxyl equivalent weight preferably is about 2500 or lower and more preferably about 2000 or lower. Generally, the hydroxy equivalent weight will be about 300 or higher, preferably about 400 or higher, and more preferably about 500 or higher. The equivalent weight may be for example between 400 and 2000.

The amine equivalent weight preferably is about 2000 or lower. Generally, the amine equivalent weight will be about 1500 or lower, preferably about 1000 or lower, and more preferably about 600 or lower. The amine equivalent weight preferably will be about 150 or higher, preferably about 200 or higher. The equivalent weight may be for example between 150 and 1000, or 200 and 1500.

The total hydroxyl and amine value can be measured in standard ways, wherein the hydroxyl and amine groups are reacted with an excess of acetic acid anhydride, and the resulting free acetic acid group is back titrated with KOH to assess the total millimolar amount of hydroxy and amine groups in 1 gram of sample. The amine value can also be assessed by titration with 0.1 N hydrochloric acid (ASTM D2572-91) and thereafter calculated back to mg KOH. The hydroxyl value can be calculated by their theoretical molar amount by subtracting the amine value from the total amine and hydroxyl value.

The amino and hydroxy-functional compound can be made in a plurality of ways, which may be dependent on the type of (polymer) backbone.

In a preferred process for the preparation of a polyester based amino and hydroxy-functional compounds, an unsaturated hydroxyl functional polyester is prepared (in one or more synthetic steps), which comprises maleate or fumarate unsaturation. The maleate and/or fumarate unsaturation is used to prepare an aspartate through addition of an aliphatic or an aromatic primary amine compound.

In a preferred process for the preparation of an acrylic (Formula II) or non-ester-type (Formula III) based amino and hydroxy-functional compounds, a hydroxyl functional acrylic polymer or any other polyol is prepared or purchased, which is, then, reacted with maleic acid anhydride to form the corresponding half-acid half-ester compound. The resultant free acid groups preferably are converted to esters by reaction with an epoxy compound. The maleate and/or fumarate unsaturation can be, like in the case of the unsaturated polyester, reacted with an amine to give an aspartic ester moiety.

Yet, in another preferred process for the preparation of acrylic (Formula II) or non-ester-type (Formula III) based amino and hydroxy-functional compounds, an epoxy-functional compound is reacted with a maleate mono-ester to form a polyol with maleate unsaturation. For example, maleic anhydride is reacted with an alcohol to form the corresponding half acid-ester. The resultant carboxylic acid compound is reacted with an epoxy-functional acrylic polymer or any other epoxy-functional compound to form the corresponding maleate and/or fumarate functional polyol. The epoxy compound can be prepared or purchased. The maleate and/or fumarate unsaturation can be, like in the case of the unsaturated polyester, reacted with an amine to give an aspartic ester moiety.

The invention furthermore relates to curable compositions comprising said amino and hydroxy-functional compounds and polyisocyanates, wherein the amount of isocyanate is present in about 60% of the molar amount of the amino and alcohol groups (isocyanate reactive group), or more.

Preferably, the amount of isocyanate is about 70% of the molar amount of the amino groups and alcohol groups or more, and even more preferable about 80% or more. Generally, the amount is about 140% or less, preferably 110% or less. The most preferred amount is about the same amount of isocyanate and isocyanate reactive groups.

The coating composition contains at least the amino and hydroxy-functional component of the invention and an isocyanate compound. However, several other components may be present, such as (a) other binder components, (b) non-reactive diluents, (c) coloring agents, (d) catalysts, flow agents, and other commonly used additives.

The other binder components may be further polymers, reactive diluents and non-reactive polymers, where introducing the latter compounds would result in the formation of an interpenetrating network (IPN). Suitable other binder polymeric additives comprise polyester polyols, polyacrylic polyols, polyether polyols and the like. Aspartate ester functional components other than already described, but for example described in the prior art may be used in admixture with the amino and hydroxy-functional compounds of the present invention.

As non-reactive diluents (or solvents), the conventional organic solvents can be used. The coating composition can be formulated as a high solid coating with relatively low amount of solvents and/or other volatile organic compounds (VOCs). Generally, the amount of VOCs is about 20 parts by weight (pbw) of the total coating composition or less, preferably about 10 pbw or less and more preferably is about 5 pbw or less.

As coloring agents, the conventional pigments, extenders, and dyes can be used, preferably in a pigment dispersion in which the pigment is stabilized for dispersion into an organic coating composition. Suitable pigments include organic and inorganic pigments. The inorganic pigments include titanium dioxide, iron oxides, zinc oxide, other metal oxides and carbon black. The organic pigments include phthalocyanine blue and green pigments, perylenes, pyrrole, arylides, indanthrones, magenta, and quinacridone red, and many other pigments. The color pigment may be chosen from those disclosed by HERBST et al., Industrial Organic Pigments, Production, Properties, Applications; 3rd Edition, Wiley-VCH, 2004, ISBN 3527305769 which is hereby incorporated by reference in its entirety. Suitable extenders include calcium carbonate, talc, barium sulfate, hydrated aluminum silicate (Pyrophyllite), calcium metasilicate (Wollastonites), kaolin clays, and other fillers.

Suitable additives comprise catalysts [such as for example Tin (IV) compounds] thixotropes, defoamers, pigment dispersants, flow agents, extenders, dehydrating agents (like molecular sieves) and the like.

The coating composition generally comprises the following components in parts by weight (pbw):

(a) aminopolyol of the invention (1-80 pbw)
(b) polyisocyanate compound (1-65 pbw)
(c) other binder constituents (0-60 pbw)
(d) colorants (0-40 pbw)
(e) additives (0-10 pbw)
(f) catalysts (0-1 pbw)
(g) solvents (0-30 pbw)
In which components (a)-(f) together are 100.

In a preferred embodiment, component (c) is present in an amount between 1 and 60 pbw, and wherein component c comprises one or more of:
i. hydroxy functional acrylic polymers, hydroxy functional polyester, hydroxy functional reactive diluent, hydroxy functional polyether, hydroxy functional polycarbonate or hydroxy functional polyurethane;
ii. non-functional or lightly functional polymers with a functionality; equivalent weight of about 5000 or higher; and aspartate functional compounds other than compound (a)
iii. and aspartate functional compounds other than compound (a).

In a further preferred embodiment, component (c) is present in an amount between 1 and 60 pbw, preferably between 5 and 50 pbw.

In a further preferred embodiment, component (c) comprises a hydroxy functional acrylic polymer.

In yet a further embodiment, component (c) comprises aspartate functional compound other than component (a).

In a further preferred embodiment, the invention provides a high-solid coating composition, wherein component (g) is present in about 10 pbw relative to components (a) through (f) or less.

In general, hydroxy-groups having the least steric hindrance around the hydroxyl groups react faster with isocyanate to form urethane linkage than sterically hindered hydroxyl groups. Thus, primary hydroxyl groups react with NCO faster than secondary and even much faster than tertiary hydroxyl groups.

It is especially preferred to utilize hydroxy-functional compounds such as polyesters, acrylics, and/or low molecular weight polyols, other polymers such as polyethers, polyurethanes, polycaprolactones, etc. are also suitable for the preparation of amino and hydroxy-functional compounds of the present invention. Preferably the hydroxy-functional polymer will have a number average molecular weight of at least about 100. Typical number average molecular weights will range from about 100 to about 10,000, and especially 100 to about 3,000. In order to control the duration of the pot-life of the final 2-component coatings, and thus the rate of viscosity increase, it is preferred in the practice of this invention to utilize hydroxy-functional compounds having either primary and/or secondary and even tertiary hydroxyl functionality. The more the steric hindrance of the hydroxyl group the slower the rate of cure will be.

One of the reactive polymers can be an amine and hydroxyfunctional polyester as described above.

Representative hydroxy-functional polyesters include those described below:

Hydroxy-functional polyesters are those prepared by condensation polymerization reaction techniques as are well known in the art. Representative condensation polymerization reactions include polyesters prepared by the condensation of polyhydric alcohols and polycarboxylic acids or anhydrides, with or without the inclusion of drying oil, semi-drying oil, or non-drying oil fatty acids. By adjusting the stoichiometry of the alcohols and the acids while maintaining an excess of hydroxyl groups, hydroxy-functional polyesters can be readily produced to provide a wide range of desired molecular weights, unsaturation content and performance characteristics.

The polyester polyols are derived from one or more aromatic and/or aliphatic polycarboxylic acids, the anhydrides thereof, and one or more aliphatic and/or aromatic polyols. The carboxylic acids include the saturated and unsaturated polycarboxylic acids and the derivatives thereof, such as maleic acid, fumaric acid, succinic acid, adipic acid, azelaic acid, dicyclopentadiene dicarboxylic acid, hexahydrophthalic anhydride, methyl-hexahydrophthalic anhydride, aromatic polycarboxylic acids, such as phthalic acid, isophthalic acid, terephthalic acid, etc. Anhydrides such as maleic anhydride, phthalic anhydride, trimellitic anhydride, or Nadic Methyl Anhydride (brand name for methylbicyclo[2.2.]heptene-2,3-dicarboxylic anhydride isomers) can also be used.

Representative saturated and unsaturated polyols which can be reacted in stoichiometric excess with the carboxylic acids to produce hydroxy-functional polyesters include diols such as ethylene glycol, dipropylene glycol, 2,2,4-trimethyl 1,3-pentanediol, neopentyl glycol, 1,2-propanediol, 1,4-butanediol, 1,3-butanediol, 2,3-butanediol, 1,5-pentanediol, 1,6-hexanediol, 2,2-dimethyl-1,3-propanediol, 1,4-cyclohexanedimethanol, 1,2-cyclohexanedimethanol, 1,3-cyclohexanedimethanol, 1,4-bis(2-hydroxyethoxy)cyclohexane, trimethylene glycol, tetramethylene glycol, pentamethylene glycol, hexamethylene glycol, decamethylene glycol, diethylene glycol, triethylene glycol, tetraethylene glycol, norbornylene glycol, 1,4-benzenedimethanol, 1,4-benzenediethanol, 2,4-dimethyl-2-ethylenehexane-1,3-diol, 2-butene-1,4-diol, and polyols such as trimethylolethane, trimethylolpropane, trimethylolhexane, triethylolpropane, 1,2,4-butanetriol, glycerol, pentaerythritol, dipentaerythritol, etc.

Typically, the reaction between the polyols and the polycarboxylic acids is conducted at about 120° C. to about 220° C. in the presence or absence of an esterification catalyst such as dibutyl tin oxide.

Additionally, hydroxy-functional polyesters can be prepared by substituting some or all of the polyols described above with epoxides and/or polyepoxides where acids and anhydride can open the oxirane ring to form the corresponding ester and hydroxy groups. Representative polyepoxides include those prepared by condensing a polyhydric alcohol or polyhydric phenol with an epihalohydrin, such as epichlorohydrin, usually under alkaline conditions. Some of these condensation products are available commercially under the designations EPON or DER from Hexion Specialty Chemicals or Dow Chemical Company, respectively, and methods of preparation are representatively taught in U.S. Pat. Nos. 2,592,560; 2,582,985 and 2,694,694 which are hereby incorporated by reference in their entirety.

If epoxy compounds are used during the preparation of hydroxy-functional polyesters, cycloaliphatic epoxies are the preferred epoxies. Commercial examples of representative preferred cycloaliphatic epoxies include 3,4-epoxycyclohexylmethyl 3,4-epoxycyclohexane carboxylate (e.g. “ERL-4221” from Dow Chemical); bis(3,4-epoxycyclohexylmethyl)adipate (e.g. “ERL-4299” from Dow Chemical); 3,4-epoxy-6-methylcyclohexylmethyl 3,4-epoxy-6-methylcyclohexane carboxylate (e.g. “ERL-4201” from Dow Chemical); bis(3,4-epoxy-6-methylcyclohexylmethyl)adipate (e.g. “ERL-4289” from Dow Chemical); bis(2,3-epoxycyclopentyl)ether (e.g. “ERL-0400” from Dow Chemical); dipentene dioxide (e.g. “ERL-4269” from Dow Chemical); 2-(3,4-epoxycyclohexyl-5,5-spiro-3,4-epoxy)cyclohexane-metadioxane (e.g. “ERL-4234” from Dow Chemical). Other commercially available cycloaliphatic epoxies are available from Ciba-Geigy Corporation such as CY 192, a cycloaliphatic diglycidyl ester epoxy resin having an epoxy equivalent weight of about 154. The manufacture of representative cycloaliphatic epoxies is taught in various patents including U.S. Pat. Nos. 2,884,408, 3,027,357 and 3,247,144, all of which are incorporated by reference in their entirety.

Other polyepoxides potentially useful in the practices of this invention include aliphatic and aromatic polyepoxies, such as those prepared by the reaction of an aliphatic polyol or polyhydric phenol and an epihalohydrin. Other useful epoxies include epoxidized oils and acrylic polymers derived from ethylenically unsaturated epoxy-functional monomers such as glycidyl acrylate or glycidyl methacrylate in combination with other copolymerizable monomers such as those listed below.

Another method to form particularly preferred hydroxy-functional polyesters comprises chain extending the hydroxyl-functional polyesters by reacting the hydroxyl groups of a (precondensed) polyester with chain extenders, preferably polyalkylene oxide or lactones such as polyethylene oxide, polypropylene oxide or caprolactone, valerolactone, and butyrolactone.

Monocarboxylic acids can be used for the preparation of hydroxy-functional polyesters to control molecular weight, functionality, and other characteristic properties. The monocarboxylic acids can be aliphatic, cycloaliphatic, aromatic or mixtures thereof. Preferably, the monocarboxylic acid contains 6 to 18 carbon atoms, most preferably 7 to 14 carbon atoms, such as octanoic acid, 2-ethylhexanoic acid, isononanoic acid, decanoic acid, dodecanoic acid, benzoic acid, hexahydrobenzoic acid, and mixtures thereof.

Monohydroxy compounds can be used in the practice of this invention to control molecular weight, functionality, and other characteristic properties. Examples of suitable monofunctional alcohols include alcohols with 4-18 carbon atoms such as 2-ethyl butanol, pentanol, hexanol, dodecanol, cyclohexanol and trimethyl cyclohexanol.

Hydroxy-functional acids can be used to replace some and/or all of the acids and polyols described above. Typical hydroxy acids that can be used include dimethylol propionic acid, hydroxypivalic acid, and hydroxystearic acid.

Representative hydroxy-functional acrylic polymers include those described below.

The acrylic polymer, in its use either as backbone for the amino and hydroxy-functional acrylic polymers, or as further binder polymer, may contain hydroxyl-functional monomers. Useful hydroxy-functional polymers can be conveniently prepared by free radical polymerization techniques such as in the production of acrylic resins. Also, the teaching of the European Patent Application EP1784435, (USPTO publication number: 20080114125) which is hereby incorporated by reference in its entirety, can be used here to prepare hydroxy-functional acrylic polymers. The polymers are typically prepared by the addition polymerization of one or more monomers. At least one of the monomers will contain, or can be reacted to produce, a reactive hydroxyl group. Other monomers which can be used in the polymerization include for example monomers having ethylenic unsaturation such as: esters of acrylic, methacrylic, crotonic, tiglic, or other unsaturated acids, vinyl compounds, styrene based materials, allyl compounds and/or other free-radically copolymerizable unsaturated monomers.

Representative hydroxy-functional monomers include 2-hydroxyethyl acrylate, 2-hydroxypropyl acrylate, 4-hydroxybutyl methacrylate, 2-hydroxypropyl methacrylate, 3-hydroxybutyl acrylate, 4-hydroxypentyl acrylate, 2-hydroxyethyl ethacrylate, 3-hydroxybutyl methacrylate, 2-hydroxyethyl chloroacrylate, diethylene glycol methacrylate, tetraethylene glycol acrylate, para-vinyl benzyl alcohol, etc.

Typically the hydroxy-functional monomers would be copolymerized with one or more monomers having ethylenic unsaturation such as: esters of acrylic, methacrylic, crotonic, tiglic, or other unsaturated acids such as: methyl acrylate, ethyl acrylate, propyl acrylate, isopropyl acrylate, butyl acrylate, isobutyl acrylate,2-ethylhexyl acrylate, amyl acrylate, laurylacrylate, nonylacrylate, decylacrylate 3,5,5-trimethylhexyl acrylate, trifluoroethyl acrylate methyl methacrylate, ethyl methacrylate, propyl methacrylate, butyl methacrylate, 2-ethylhexyl methacrylate, laurylmethacrylate, nonylmethacrylate, decylmethacrylate isobornyl methacrylate, cyclohexyl methacrylate, dimethylaminoethyl methacrylate, ethyl tiglate, methyl crotonate, ethyl crotonate, etc.

Suitable vinyl compounds include vinyl acetate, vinyl propionate, vinyl butyrate, vinyl isobutyrate, vinyl benzoate, vinyl m-chlorobenzoate, vinyl p-methoxybenzoate, vinyl alpha-chloroacetate, vinyl toluene, vinyl chloride, α-olefins, vinyl esters of α,α-branched monocarboxylic acid (C₉-C₁₀) such as VeoVa® 9 and VeoVa® 10 Hexion etc. Suitable styrene-based materials include styrene, vinyl toluene, alpha-methyl styrene, alpha-ethyl styrene, alpha-bromo styrene, 2,6-dichlorostyrene, etc. Suitable allyl compounds include allyl chloride, allyl acetate, allyl benzoate, allyl methacrylate, etc. Suitable other copolymerizable unsaturated monomers include acrylonitrile, methacrylonitrile, dimethyl maleate, isopropenyl acetate, isopropenyl isobutyrate, acrylamide, methacrylamide, and dienes such as 1,3-butadiene, etc.

The polymers are conveniently prepared by conventional free radical addition polymerization techniques. Frequently, the polymerization will be initialized by conventional initiators known in the art to generate a free radical such as azobis(isobutyronitrile), tert-butyl peroxy-2-ethylhexanoate, dibutyl peroxide, cumene hydroperoxide, t-butyl perbenzoate, etc. Typically, the acrylic monomers are heated in the presence of the initiator at temperatures ranging from about 35° C. to about 220° C., and preferably 120° C. to 200° C., to affect polymerization. The molecular weight of the polymer can be controlled, if desired, by the monomer selection, reaction temperature, pressure and time, and/or the use of chain transfer agents as is well known in the art.

Representatives of other hydroxy-functional polymers include those described below.

Polyether polyols are well known in the art and are conveniently prepared by the reaction of a diol or polyol with the corresponding alkylene oxide. These materials are commercially available and may be prepared by a known process such as, for example, the processes described in Encyclopedia of Chemical Technology, Volume 7, pages 257-262, published by Interscience Publishers, Inc., 1951 which is hereby incorporated by reference in its entirety. Representative examples include the polypropylene ether glycols and polyethylene ether glycols, like PPG200, PPG400, PPG1000 and the like. Other suitable hydroxy-functional polymers include polyvinylalcohol, polyallylstyrenealcohol, partially saponified polyvinylacetate and the like.

Other useful hydroxy-functional polymers can be hydroxy-functional polyurethanes. These can be prepared by the reaction of at least one polyol, such as those representatively described above, with polyisocyanates to produce hydroxy-functional urethanes. Representative polyisocyanates having two or more isocyanate groups per molecule include the aliphatic compounds such as ethylene, trimethylene, tetramethylene, pentamethylene, hexamethylene, 1,2-propylene, 1,2-butylene, 2,3-butylene, 1,3-butylene, ethylidene and butylidene diisocyanates; the cycloalkylene compounds such as 3-isocyanatomethyl-3,5,5-trimethylcyclohexylisocyanate, and the 1,3-cyclopentane, 1,3-cyclohexane, and 1,2-cyclohexane diisocyanates; the aromatic compounds such as m-phenylene, p-phenylene, 4,4′-diphenyl, 1,5-naphthalene and 1,4-naphthalene diisocyanates; the aliphatic-aromatic compounds such as 4,4′-diphenylene methane, 2,4- or 2,6-toluene, or mixtures thereof, 4,4′-toluidine, and 1,4-xylylene diisocyanates; the nuclear substituted aromatic compounds such as dianisidine diisocyanate, 4,4′-diphenylether diisocyanate and chlorodiphenylene diisocyanate; the triisocyanates such as triphenyl methane-4,4′,4″-triisocyanate, 1,3,5-triisocyanate benzene and 2,4,6-triisocyanate toluene; and the tetraisocyanates such as 4,4′-diphenyl-dimethyl methane-2,2′-5,5′-tetraisocyanate; the polymerized polyisocyanates such as tolylene diisocyanate dimers and trimers, and other various polyisocyanates containing biuret, urethane, and/or allophanate linkages. The polyisocyanates and the polyols are typically reacted at temperatures of 25° C. to about 150° C. to form the hydroxy-functional polymers.

Any of the above hydroxy-functional compounds described above can be reacted with maleic anhydride, substituted maleic anhydride, or any other unsaturated dicarboxylic acid derivative or acid anhydrides, to form the half acid-ester at a temperature between 40° to 80° C. The stoichiometry of the anhydride to the hydroxyl can be varied from 1 to 0.05 molar ratios depending on the desired excess of the hydroxyl groups over the amine functionality of the final amino and hydroxy-functional polymers. The resultant half acid-ester can be reacted with monoepoxide to an acid value of less than 5 to form the corresponding unsaturated polymer or oligomer.

Representative useful monoepoxides include the monoglycidyl ethers of aliphatic or aromatic alcohols such as butyl glycidyl ether, octyl glycidyl ether, nonyl glycidyl ether, decyl glycidyl ether, dodecyl glycidyl ether, p-tert-butylphenyl glycidyl ether, and o-cresyl glycidyl ether. Monoepoxy esters such as the glycidyl ester of versatic acid (commercially available and known as CARDURA E from Hexion Chemicals Company), or the glycidyl esters of other acids such as tertiary-nonanoic acid, tertiary-decanoic acid, tertiary-undecanoic acid, etc. are also useful. Similarly, if desired, unsaturated monoepoxy esters such as glycidyl acrylate, glycidyl methacrylate or glycidyl laurate could be used. Additionally, monoepoxidized oils can also be used.

Other useful monoepoxies include styrene oxide, cyclohexene oxide, ethylene oxide, propylene oxide, 1,2-butene oxide, 2,3-butene oxide, 1,2-pentene oxide, 1,2-heptene oxide, 1,2-octene oxide, 1,2-nonene oxide, 1,2-decene oxide, and the like.

In general, the reaction of primary amines with maleate or fumarate esters produce aspartate groups with different degree of reactivity toward NCO. The higher the steric hindrance around the nitrogen atom of the amine, the slower the reactivity toward NCO and visa versa.

Primary amines useful for the present invention include, but not limited to, various alkyl, aryl or aralkyl amines having 1-30 carbon atoms in the molecule. Specific examples for the alkyl amine include methylamine, ethylamine, propyl- and isopropylamine, butyl-isobutyl- and teriarybutylamine, 1,3-dimethyl- and 3,3-dimethylbutylamine, pentyl-isopentyl-tertiaryamy, and neopentylamine, hexylamine isomers, cyclohexylamine, 2-methylcyclohexylamine isomers, 4-methylcyclohexylamine isomers, cycloheptylamine, heptylamine isomers, octylamine isomers, nonylamine, dodecvylamine, stearylamine, cyclohexylmethylamine, α-methylcyclohexanemethylamine. Examples of aryl amines include aniline, toluidine isomers, aniline, dimethylaniline isomers, ethylaniline isomers, propyl- and isopropylaniline isomers, 2,6-diethylaniline, and various substituted anilines. Examples of aralakyl amines include benzyl amine, α-methylbenzylamine, α-ethylbenzylamine, 4, α-dimethylbenzylamine, phenethylamine, alkyl phenethylamine isomers, and 4-phenylbutylamine. Other amines suitable for the present invention are amino polyols such as 1-aminohydroxyprpoane isomers, 2-amino-2-methyl-1,3-propanediol, 2-amino-2-ethyl-1,3-propanediol, 2-amino-2-hydroxymethyl-1,3-propanediol, and alkyl esters of amino acids such as methyl, ethyl, propyl and butyl esters of glycine, alanine, phenylalanine, leucine, isoleucine, aspartic acid, glutamic acid, and valine.

The coating composition of the invention comprises (optionally blocked) isocyanate-functional cross-linkers. These compounds are based on the usual isocyanate-functional compounds known to a person skilled in the art. More preferably, the coating composition comprises cross-linkers with at least two isocyanate groups. Examples of compounds comprising at least two isocyanate groups are aliphatic, alicyclic, and aromatic isocyanates such as hexamethylene diisocyanate, 2,4,4-trimethyl hexamethylene diisocyanate, dimeric acid diisocyanate, such as DDI® 1410 ex Henkel, 1,2-cyclohexylene diisocyanate, 1,4-cyclohexylene diisocyanate, 4,4′-dicyclohexylene diisocyanate methane, 3,3′-dimethyl-4,4′-dicyclohexylene diisocyanate methane, norbornane diisocyanate, m- and p-phenylene diisocyanate, 1,3- and 1,4-bis(isocyanate methyl)benzene, 1,5-dimethyl-2,4-bis(isocyanate methyl)benzene, 2,4- and 2,6-toluene diisocyanate, 2,4,6-toluene triisocyanate, α,α,α′,α′-tetramethyl o-, m-, and p-xylylene diisocyanate, 4,4′-diphenylene diisocyanate methane, 4,4′-diphenylene diisocyanate, naphthalene-1,5-diisocyanate, isophorone diisocyanate, 4-isocyanatomethyl-1,8-octamethylene diisocyanate, the isocyanates described for the preparation of hydroxyfunctional urethanes above, and mixtures of the aforementioned polyisocyanates.

Other (optionally blocked) isocyanate compounds are based on adducts of polyisocyanates, e.g., biurets, isocyanurates, imino-oxadiazinediones, allophanates, uretdiones, and mixtures thereof. Examples of such adducts are the adduct of two molecules of hexamethylene diisocyanate or isophorone diisocyanate to a diol such as ethylene glycol, the adduct of 3 molecules of hexamethylene diisocyanate to 1 molecule of water, the adduct of 1 molecule of trimethylol propane to 3 molecules of isophorone diisocyanate, the adduct of 1 molecule of pentaerythritol to 4 molecules of toluene diisocyanate, the isocyanurate of hexamethylene diisocyanate, available from Bayer under the trade designation Desmodur® N3390, a mixture of the uretdione and the isocyanurate of hexamethylene diisocyanate, available from Bayer under the trade designation Desmodur® N3400, the allophanate of hexamethylene diisocyanate, available from Bayer under the trade designation Desmodur® LS 2101, and the isocyanurate of isophorone diisocyanate, available from Evonik, under the trade designation Vestanat® T1890. Furthermore, (co)polymers of isocyanate-functional monomers such as α,α′-dimethyl-m-isopropenyl benzyl isocyanate are suitable for use. Finally, as is known to the skilled person, the above-mentioned isocyanates and adducts thereof may be at least partly present in the form of blocked isocyanates.

For blocking the polyisocyanates it is possible in principle to employ any blocking agent which can be employed for the blocking of polyisocyanates and has a sufficiently low deblocking temperature. Blocking agents of this kind are well known to the skilled worker and need not be elucidated further here. It is possible to employ a mixture of blocked polyisocyanates which contains both isocyanate groups blocked with a first blocking agent and isocyanate groups blocked with a second blocking agent. Reference is made to WO 98/40442 which is hereby incorporated by reference in its entirety.

All patent applications and patents cited herein are herein incorporated by reference.

The invention will be further elucidated with the following non-limiting examples:

### EXAMPLES

### A) Preparation of Amino Polyester Polyols

Example 1 is an example of an amino acrylic polyol with 4.3 OH groups and 1 NH group per molecule.

### Example 1-A

### Preparation of Epoxy Functional Acrylic Polyols (EAPO)

A one gallon jacketed pressure reactor equipped with a condenser, agitator, heating jacket; addition funnel, thermocouple attached to a temperature control, nitrogen pressure controller and attached to pressure transducer is charged with 17.3 parts (by weight) of methyl amyl ketone. The temperature of the reactor is set at 182 deg C. at 68 psi nitrogen pressure. At this temperature and pressure, a mixture of 26.4 parts (by weight) styrene, 14.1 parts butyl acrylate, 21.6 parts butyl methacrylate, 11.8 parts hydroxyl propyl methacrylate, 4.0 parts glycidyl methacrylate, 1.4 parts methyl methacrylate, 1.9 parts 2,3-epoxypropylneodecanoate (Cardura-E) and 0.9 parts di-tertiarybutyl peroxide was added over three hours. After three hours the reaction was held for 30 min. Then the temperature was decreased to 138 deg C. and pressure was released to 15 psi (atmosperic pressure). At 138 deg C., a blend of 0.4 parts tert-butylperoxybenzoate and 0.3 parts methylamylketone was added for 30 min. The reaction was held for 30 min at this temperature. The epoxy-functional acrylic polyol (EAPO) was filtered through 25 micron bag and stored. The obtained EAPO has a non-volatile material of 82% (measured at 130° C. forced air oven for 1 h); an acid value of 0.43; a hydroxyl value of 77; a hydroxy equivalent wt. of 890; a number average molecular weight (Mn) of 2250; a weight average molecular weight (Mw) of 4900; and a polydispersity of 2.2.

### Example 1-B

### Preparation of Monobutyl Maleate

A four-necked reaction flask equipped with a condenser, agitator, heating mantle; addition funnel, thermocouple attached to a temperature control, and Dean-Stark trap primed with methylamylketone, is charged with 57.0 parts (by weight) of maleic anhydride and 43.0 parts (by weight) of 1-butanol and heated under 0.5 SCFH (standard cubic feet per hour) (0.014 m³ hr⁻¹) nitrogen flow to 50° C. The progress of the reaction was monitored by fourier transform infrared spectroscopy (FT-IR). The reaction was carried out at this temperature till the disappearance of anhydride peaks, 1776 and 1851 cm⁻¹. The obtained monobutyl maleate is stored in a glass reactor. The unsaturated monobutyl maleate is filtered and stored.

It has a non-volatile material of 100%; an acid value of 326; an unsaturated equivalent wt. of 172; a number average molecular weight (Mn) of 262; a weight average molecular weight (Mw) of 271; and a polydispersity of 1.03.

### Example 1-C

### Preparation of 100% NVM Unsaturated Polyester Polyols

A four-neck reaction flask equipped as in Example 1-B, is charged with 95.5 parts (by weight) of expoxy acrylic polyol (EAPO of Example 1-A). 4.5 parts of monobutyl maleate (Example 1-B) was added slowly over one hour. After the addition, the reaction was heated under 0.5 SCFH (standard cubic feet per hour) (0.014 m³ hr⁻¹) nitrogen flow to 125° C. and maintained at such temperature until an acid value of less than 2 is attained. The unsaturated acrylic polyol (UAPO) is filtered and stored.

The obtained UAPO has a non-volatile material of 82.3% (measured by mixing the resin with NCO at NCO/OH of 1:1 and heated for 1 hr. at 130° C.); an acid value of 1.6; a hydroxyl value of 77; a hydroxyl equivalent weight of 725, an unsaturated equivalent wt. of 3105; a number average molecular weight (Mn) of 2200; a weight average molecular weight (Mw) of 5700; and a polydispersity of 2.6.

### Example 1-D

### Preparation of Amino Acrylic Polyol (AAPO)

A four-neck reaction flask equipped as in Example 1-B, is charged with 98.2 parts of UAPO from Example 1-C. Sec-butyl amine (1.8 parts) is added drop-wise via addition funnel over 1 hour while maintaining the temperature at or below 50° C. Heating is continued overnight at 50° C. The resultant light yellow viscous liquid obtained is filtered. The resulting resin has a non-volatile material (NVM) of 82.5%; a viscosity of 85000 m Pa·s; a number average molecular weight (Mn) of 2100; a weight average molecular weight (Mw) of 5400; and a polydispersity of 2.6. The hydroxyl equivalent weight is 787 (OH value=71), and the amine equivalent weight is 3373 (amine value=17) and the average equivalent weight for the total functionality is 638. The amine value is determined by titrating the amine with 0.1 N hydrochloric acid (ASTM method D2572-91). The total hydroxyl plus the amine value is determined by acetylating both the hydroxy and amine groups with acetic acid anhydride and then titrating acetic acid with KOH. The hydroxyl value is then determined by subtracting the amine value from the total amine plus hydroxyl values.

### Example 2

Example 2 is an example of for the preparation of polyester with on average 1 hydroxyl groups and 4 NH groups per molecule.

### Example 2-A

### Preparation of Unsaturated Polyester Polyol

A four-necked reaction flask equipped with a condenser, agitator, heating mantle; addition funnel, thermocouple attached to a temperature control, and Dean-Stark trap primed with xylene, is charged with 45.25 parts of neopentyl glycol (NPG), 5.69 parts of trimethylol propane (TMP), 37.35 maleic anhydride (MAn), 20.95 parts isononanic acid, 0.14 part of triphenyl phosphate, and 0.05 part of Fascat 9100, an unsaturated polyester polyol (UPPO) is obtained. This UPPO has a non-volatile material of 99% (measured by mixing the resin with NCO at NCO/OH of 1:1 and heated for 1 hr. at 130° C.); an acid value of 1.0; a hydroxyl value of 125; a number average molecular weight (Mn) of 1150; a weight average molecular weight (Mw) of 1800; and a polydispersity of 1.6.

### Example 2-B

### Preparation of Amino Polyester Polyol (APPO)

A four-neck reaction flask equipped as in Example 2-A, is charged with 69.1 parts of UPPO from Example 2-A, and 30.9 parts α-Methylbenzylamine a viscous yellow resin of amino polyester polyol (APPO) is obtained. The APPO has a non-volatile material (NVM) content of 99.75%; a viscosity of 435 m Pa·s; a Mn of 1200; a Mw of 1900; and a polydispersity of 1.6. This APPO has a hydroxyl equivalent weight of 1452, an amine equivalent weight of 380 and an average equivalent weight for the total functionality of 301.

### Example 3

Example 3 is an example of the preparation of polyester with on average 1 hydroxyl groups and 2 NH groups per molecule.

### Example 3-A

### Preparation of Unsaturated Polyester Polyol

The procedure of Example 2-A is used for this example. Thus from 44.17 parts of neopentyl glycol (NPG), 5.02 parts of trimethylol propane (TMP), 11.46 parts of adipic acid (AdA), 25.00 maleic anhydride (MAn), 24.55 parts isononanoic acid, 0.14 part of tirphenyl phosphate, and 0.05 part of Fascat 9100, a viscous UPPO resin is obtained. The unsaturated polyester polyol (UPPO) is cooled to 150° C. and filtered.

The resulting UPPO has a non-volatile material of 99%, an acid value of 1.0; a hydroxyl value of 125; a number average molecular weight (Mn) of 1177; a weight average molecular weight (Mw) of 2800, a hydroxyl equivalent weight of 732, an unsaturation equivalent weight of 392.

### Example 3-B

### Preparation of Amino Polyester Polyol (APPO)

The procedure of Example 2-B is used for this example. Thus from 100 parts of UPPO from Example 3-A, and 19.1 parts α-Methylbenzylamine a viscous yellow resin of amino polyester polyol (APPO) is obtained. The resultant light yellow viscous liquid obtained is filtered. The resulting resin has 97.6% non-volatile material (NVM); a Mn of 1300; a Mw of 2950; a hydroxyl equivalent weight of 938, an amine equivalent weight of 502, and an average equivalent weight of 327.

### Example 4

### B) Preparation of Amino Acrylic Polyols

Example 4 is an example of for the preparation of amino acrylic polyol (AAPO) having on average 3.5 hydroxyl groups and 3.5 NH groups per molecule.

### Example 4-A

### Preparation of Low MW Acrylic Polyols

In a 6.5-liter stainless steel pressure reactor equipped with heating and cooling systems, mechanical stirrer, metering pumps, pressurized liquid and gas inlets and outlets, and pressure and temperature gauges is charged with 37 parts of n-butyl acetate, pressurized to 55 psi and heated to 195° C. A mixture of 34.4 parts of 2-hydroxyethyl acrylate (2-HEA), 2 parts of 2-hydroxyethyl methacrylate (2-HEMA), 29.1 parts of iso-butyl methacrylate (IBMA), 22.5 parts of methyl methacrylate (MMA), 10.8 parts of isobornyl methacrylate (IBOMA), 1 part methacylic acid (MAA), and 1.62 parts of di-t-butyl peroxide (Trigonox B from Akzo Nobel Polymer Chemicals) is added to the reactor over a period of 3.0 hours. After an additional 30 minutes, the mixture is cooled to 120° C. A mixture of 4.5 parts of butyl acetate and 0.5 part of t-butyl peroctoate (Trigonox 21 from Akzo Nobel Polymer Chemicals) is added over 75 minutes at 125° C. The reactor content is cooled to 65-70° C. and vacuum is applied to remove volatiles by distillation. The acrylic polyol resin is filtered through 25 m bag. The resultant resin has an 81% non-volatile content (NVM), a Mn of 1118, a Mw of 1668, a hydroxyl equivalent weight of 320, a hydroxyl value of 175, a Brookfield viscosity of 192 Poise, a density of 9.17 lb/gal and an APHA color reading of 21.

### Example 4-B

### Preparation of Unsaturated Acrylic Polyols

A four-necked reaction flask equipped with a condenser, agitator, heating mantle, nitrogen inlet, and a thermocouple attached to a temperature control box is charged with 47.20 parts of acrylic polyol prepared in Example 4-A, 14.20 parts maleic anhydride, and 0.22 part of N,N-dimethylbenzyl amine. The mixture is heated to 70° C. and maintained at such temperature for 8-14 hours, or until the disappearance of anhydride as detected by Infra Red. Glycidyl versatate (33.1 parts) known as Cardura E-10 (from Hexion) is added and the reaction temperature is increased to 120-125° C. The reaction is continued until the acid value is below 2. Additional glycidyl versatate (5 parts) is added to bring the AV to the desired range of 1-2. The resultant unsaturated hydroxyl resin has a non-volatile content of 90%, hydroxyl equivalent weight of 678, unsaturation equivalent weight of 690, a Mn of 1700, a Mw of 4700 and used as is in Example 4-C.

### Example 4-C

### Preparation of Amino Acrylic Polyols

The resin of Example 4-B (88.75 parts) is added to a four necked flask equipped as in Example 3-B and with additional funnel and heated to 40° C. Cyclohexyl amine (11.25 parts; 99% purity) is added over 1 hrs period while maintaining the temperature around 50° C. but below 60°. C. Heating is continued for additional 8-20 hrs at 50° C. The disappearance of double bond is monitored by IR at 1600 cm^{˜}1. The resultant resin has a Mn of 1500, Mw of 4300, non-volatile material of 91%, a hydroxyl equivalent weight of 772, an amine equivalent weight of 786, and an average equivalent weight of 390.

### C) Preparation of Amino & Hydroxyl Functional Reactive Diluents

### Example 5

In a procedure similar to that of Examples 4-B and 4-C, the following raw materials are used to prepare amino and hydroxy functional reactive diluent. Thus from 11.1 parts hexane diol, 18.4 maleic anhydride, 0.1 part N,N-dimethylbenzylamine, 48.5 parts glycidyl versatate; and 21.9 parts α-methylbenzylamine, a viscous liquid resin obtained. The resultant resin has a non-volatile material of 99.6%, Mn of 980, Mw of 1460, a hydroxy equivalent weight of 532, amine equivalent weight of 554 and an average equivalent weight of 272.

### Example 6

This example demonstrates the effect of steric hindrance of the primary amine used on speed of cure or gel time of APPO with isocyanate.

Example 3B is reproduced using equimolar amount of 2-methylcyclohexylamine instead of α-Methylbenzyl amine Thus from 77.6 parts of unsaturated polyester from Example 3-A and 22.4 parts of 2-methylcyclohecylamine, a viscous light yellow liquid is obtained having a Mn of 1180, a Mw of 2780; a hydroxyl equivalent weight of 943, an amine equivalent weight of 506, and an average equivalent weight of 329.

### Example 7

This example demonstrates the effect of steric hindrance of the hydroxyl groups used on speed of cure or gel time of APPO with isocyanate.

The procedure of Example 3A is used here to prepare the hindered unsaturated polyesterpolyol. Thus from 52.6 parts of 2,2,4-trimethyl 1,3-pentanediol, 4.26 parts trimethylol propane, 9.7 parts adipic acid, 21.2 parts maleic anhydride and 0.2 parts triphenylphosphite, an unsaturated polyester polyol having an acid value of 7.8 is obtained. Glycildyl versatate (3 parts) is added to consume the residual acid and the reaction is heated at 125° C. for 6 hours. The resultant unsaturated polyester polyol (100 parts) is treated with α-methylbenzyl amine (25.4 parts) as described in Example 3-B. The resultant amino polyester polyol has an Mn of 1280, a Mw of 2880; a hydroxyl equivalent weight of 905, an amine equivalent weight of 597, and an average equivalent weight of 359.

### Example 8

### Example 8-A

### Preparation of Epoxy-Functional Acrylic Polymer

In a procedure similar to that described for Example 4-A, an epoxy acrylic polymer composed of 60% glycidyl methacrylate and 40% butyl acrylate is prepared at 90% solids in xylene. The resultant epoxy resin has a Mn of 970, a Mw of 1760, and an epoxy equivalent weight of 237.

### Example 8-B

### Preparation of Unsaturated Acrylic Polyols

In a reaction set-up similar to that of Example 4-B, 58 parts of epoxy acrylic polymer (Example 8-A) and 42 parts of monobutyl maleate half acid-ester is mixed at room temperature and the mixture is heated to 110° C. until acid value is stalling around 10. Additional epoxy acrylic polymer (4.5 parts) is added to bring the acid value to below 3. The monobutyl mealeate half acid-ester is prepared from maleic anhydride, n-butanol (10% excess mole ratio) and dimethylbenzyl amine (0.25% by weight) at 50° C. The reaction is carried out until no anhydride adsorption is remained in the FTIR spectrum.

### Example 8-C

### Preparation of Amino Acrylic Polyols

The procedure of Example 4-C is used here. Thus from unsaturated acrylic resin of Example 8-B (79.7 parts) and 21.3 parts of 2-methylcylcohexyl amine, there is obtained an amino and hydroxy functional acrylic polymers having the following properties: a Mn of 1564, Mw of 4830, non-volatile material of 94.5%, a hydroxyl equivalent weight of 540, an amine equivalent weight of 540, and an average equivalent weight of 270.

### D) Coating Performance of Amino & Hydroxyl Functional Compounds

White paints are made from resins prepared according to the present invention (Examples 9-17) and similar paints are made for Comparative Examples A and B. The following general procedure is used to prepare such white paints.

General Procedure for the Preparation of White Paint:

To a high speed Cowles mixer, the following ingredients are added:

19.5 parts of APPO from Example 3-B, 2.0 parts xylene, 0.47 part of MPA 4020-X (anti settling agent from Elementis), 1.24 parts methyl amyl ketone (MAK), 1.41 parts and Disperbyk 163 (pigment dispersant from BYK-Chemie) and mixed for 5 minutes at low speed. Titanium dioxide (46.8 parts, R706 from DuPont) is sifted in slowly while mixing. Methyl amyl ketone (1.24 parts) is added and the slurry is dispersed for 15 minutes at high speed or until a Hegman grind of 6.5-7 is obtained. Additional resin from Example 3-B (9.76 parts), Byk 077 (0.47 part), Byk 306 (0.09 part) and methyl isobutyl ketone (4.31 parts) are added. The TiO₂ grind base is mixed for additional 20-30 minutes at low speed and 16.67 parts Desmodur N-3390 (from Bayer) is added.

### Comparative Examples A & B

Comparative Examples A & B are made according to the general paint procedures described above for using Desmophen NH-1520 (From Bayer) as the binder for Comparative Example A and acrylic polyol, 27-1316 (2.2% OH; from Nuplex Resins), as the binder for Comparative Example B.

### Example 9 to 13

### Coatings Performance of APPO

White paints are made from resins of Examples 1-B, 2-B, and 3-B, 6 and 7 as described in the general procedures above and various tests are carried out. Various tests, listed in Table 2, are carried out according to test methods and instruments listed in Table 1. The freshly prepared paints are used to measure the Kreb viscosity and gel time. For the latter test, about 100 g of a freshly activated white paint is placed in a paper cub and the L-shaped spindle of Shyodu gel Time (Model 100, made by Paul N. Gardner, Inc.) is immersed into the paint and start to rotate. Rotation continues until the paint is gelled and the spindle has stopped. The time required for the spindle to freely rotate is called the gel time. Paints are drawn down on Bonderite B-1000 panels with Doctor blade to give a 2-2.5 mils dry film thickness and the wet panels, immediately, placed under a Gardner dry-time recorder. Gloss, hardness, impact resistance, and conical Mandrel flexibility test are performed after drying at controlled temperature and humidity (72° F. and 50% RH, respectively).

TABLE 1
Equipment & ASTM Methods used for various Coatings Tests

| TEST | INSTRUMENT | ASTM METHOD No. |
|---|---|---|
| Viscosity | BYK Gardner | D-562 |
| | Model KU1 + Stormer | |
| 20°/60° Gloss | BYK-Gardner | D-523 |
| | Model 4430 Gloss Meter | |
| Drying Time | Gardco model DT-5020 | D-5895 |
| | Dry Timer | |
| Impact Resistance | Gardner Impact Tester | D-2794 |
| Elongation | Gardner Conical Mandrel | D-522 |
| Koenig | BYK-Gardner | D-5895 |
| Pendulum Hardness | Koenig Pendulum Tester | |

Coatings results of various tests are shown in Table 2 for Examples 8-12

TABLE 2
Coatings Data for Examples 8-12

| | Example 9 | Example 10 | Example 11 | Example 12 |
|---|---|---|---|---|
| Resin Type | Example 2B | Example 3B | Example 6 | Example 7 |
| Paint Viscosity | 97 | 89 | 80 | 78 |
| (KU) | | | | |
| Gloss 20°/60° | 93/97 | 89/95 | 85/93 | 89/95 |
| VOC, g/l | 218 | 218 | 227 | 227 |

Dry Times

| | | | | |
|---|---|---|---|---|
| Dry touch | 8′ | 16′ | 15′ | 16′ |
| Tack free | 12′ | 34′ | 1 H 14′ | 1 H 10′ |
| Dry hard | 22′ | 46′ | 2 H 36′ | 3 H 08′ |
| Dry through | 38′ | 1 H 20′ | 4 H 14′ | 5 H 10′ |

KPH hardness

| | | | | |
|---|---|---|---|---|
| 1 day | 47 | 14 | 33 | 22 |
| 7 day | 86 | 39 | 49 | 42 |

Impact Resistance

| | | | | |
|---|---|---|---|---|
| 7 day (dir/rev) | 40/20 | 145/140 | 160/150 | 110/110 |
| inch. Lb. | | | | |

Elongation (Conical Mandrel)

| | | | | |
|---|---|---|---|---|
| 7 day | No Effect | No Effect | No Effect | No Effect |
| Gel Times | 16 min | 41 min | 4 H 5′ | 4 H 35′ |

### Example 13 to 16 and Comparative Examples A & B

To test the advantages of the present invention, in comparison with the prior art, blends of white paints of Example 10 and those from Comparative Examples A or B, at 50-50% by weight blend ratio, yield Examples 14 and 15, respectively. Example 16 is a paint made from AAPO of Example 4. Blend of paint of Example 15 with a comparative experiment A provides Example 16. Coatings test data for Examples 13-16 and the Comparative Examples A and B are shown in Table 3.

TABLE 3
Coatings Data for Example 13-16 and Comparative Examples A & B:

| | Example No. | | | | | |
|---|---|---|---|---|---|---|
| | | | | | Comparative | Comparative |
| | 13 | 14 | 15 | 16 | Example A | Example B |
| Resin | 50/50% | 50/50 | Example 4 | 50/50% | NH-1520* | 27-1316* |
| Type | Blend of | Blend of | | Blend of | Aspartate | Acrylic |
| | Example 3 & | Example 3 & | | Example 4 & | | Polyol |
| | NH-1520 | 27-1316 | | NH-1520 | | |
| Paint | 74KU | 92 KU | 106 | 76 | 64 KU | 72 KU |
| Viscosity | | | | | | |
| Gloss | 88/93 | 89/94 | 90/94 | 87/93 | 87/93 | 89/95 |
| 20°/60° | | | | | | |
| VOC, g/l | 197 | 226 | 218 | 217 | 215 | 333 |

Dry times

| | | | | | | |
|---|---|---|---|---|---|---|
| Dry touch | 16′ | 18′ | 7′ | 23′ | 1H 30′ | 30′ |
| Tack free | 1H | 32′ | 30′ | 55′ | 2H 40′ | 2H 45′ |
| Dry hard | 1H 40′ | 1H | 1H | 2H 17′ | 5H 50′ | 13H 30′ |
| Dry | 2H 50′ | 2H 15′ | 2H 13′ | 5H | 10H | >24H |
| through | | | | | | |

KPH hardness

| | | | | | | |
|---|---|---|---|---|---|---|
| 1 day | 81 | 14 | 13 | 46 | 149 | 14 |
| 7 day | 129 | 37 | 34 | 94 | 176 | 54 |

Impact Resistance

| | | | | | | |
|---|---|---|---|---|---|---|
| 7 day | 45/20 | 120/135 | 130/130 | 40/15 | 10/0 | 130/130 |
| (dir/rev) | | | | | | |
| inch. lb. | | | | | | |

Elongation (Conical Mandrel)

| | | | | | | |
|---|---|---|---|---|---|---|
| 7 day | No Effect | No Effect | No | No Effect | Complete | No Effect |
| | | | Effect | | delamination | |
| Gel Time | 2H, 24′ | 1H 47′ | 56′ | 3H 23′ | 11H, 8′ | >24H |

*Comparative Example A = NH-1520 a Polyaspartate Resin from Bayer
**Comparative Example B = 27-1316 an Acrylic Polyol (2.2% OH) from Nuplex Resins

Results of Tables 1 and 2 clearly demonstrate that coatings containing resins of the present invention have shorter dry-times, higher impact resistance and better elongation or flexibility than coatings based on the prior art shown in the Comparative Examples A and B.

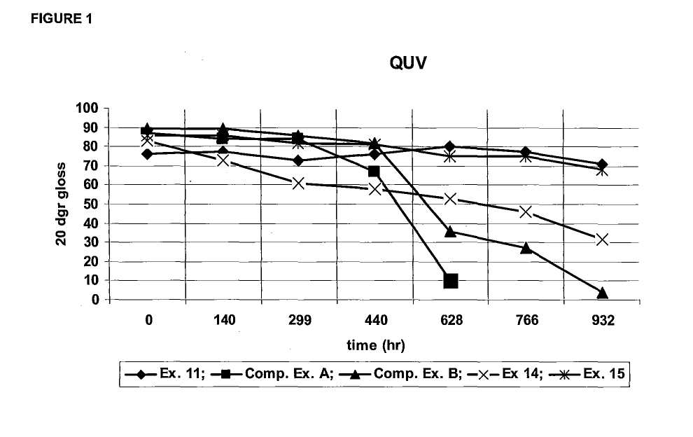

The QUV 313 accelerated weathering of white paints of Examples 11, 14, 15, and Comparative Examples A and B are shown in FIG. 1 which shows the 20° Gloss Retention of several amino and hydroxy-Functional compounds in white paint as described in several of the examples.

QUV is the name of the instrument made by the Q-Lab company. A QUV test chamber uses fluorescent lamps to provide a radiation spectrum centered in the ultraviolet wavelengths. Moisture is provided by forced condensation, and temperature is controlled by heaters. The test references that can be referred to are: ASTM D4329, D4587ASTM D4329, D4587.

The results of FIG. 1 clearly demonstrate the superior durability of the coatings based on the present invention over those of the prior art. In addition, it shows that paint made according to the present invention can improve the durability of conventional acrylic urethane coatings (Example 15).

Thus, the invention has been described by reference to certain embodiments discussed above. Further modifications in addition to those described above may be made to the structures and techniques described herein without departing from the spirit and scope of the invention. Accordingly, although specific embodiments have been described, these are examples only and are not limiting upon the scope of the invention.

What is claimed is:

1. An amino and hydroxy-functional compound, not being a polyester, wherein the amino and hydroxy-functional compound comprises an amine in the form of aspartic acid esters functionality, and wherein the amino and hydroxy-functional compound has:
(a) a molecular weight (Mn) of at least about 500;
(b) an acid value of about 5 or less;
(c) a hydroxyl value of about 30 or more; and
(d) an amine value of about 30 or more.

2. The amino and hydroxy-functional compound according to claim 1, wherein molecules of the compound have on average at least one of:
(e) at least 1 secondary amino group as an aspartate,
(f) and at least 1 hydroxy group,
and
(g) an average total functionality of about 1.8 or higher.

3. The amino and hydroxy-functional compound according to claim 1, wherein the compound further comprises a hydroxy-functional polyester in the form of a polyester polyol, and wherein the polyester has a general structure according to formula I: 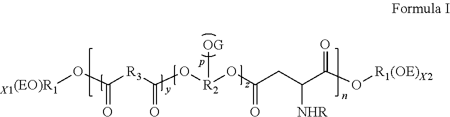
wherein:
R=mono-valent alkyl, aryl and/or arylalkyl radical;
R₁=residue obtained from a polyol after removing OH groups and wherein the R₁ has a valency of 1 to 6;
R₂=residue obtained from a polyol after removing OH groups and wherein the R₂ has a valency of 2 to 6;
R₃=divalent saturated and/or unsaturated alkyl and/or aryl radical;
E=H or acyl group having 1 to 18 carbon atoms;
X¹, X²=an integer having an equal or different values of 0 to 5, and wherein a sum of X¹ and X² is at least 1;
y, z=an integer having a value of 0 or 1;
p=an integer having a value between 0 to 4;
G=E and/or is a residue having the following structure: 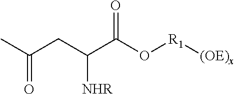
n=an integer having a value between 1 to 10.

4. The amino and hydroxy-functional compound according to claim 1, wherein the compound is in the form of an acrylic polymer and wherein the acrylic polymer has the general structure according to Formula II: 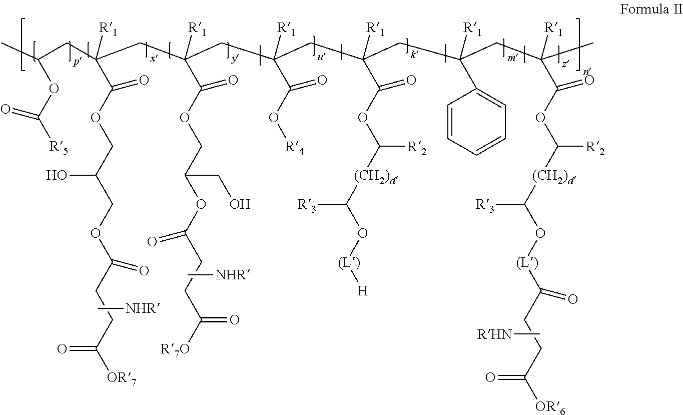
wherein:
R′=mono-valent alkyl, aryl and/or arylalkyl radical;
R′₁=H, methyl;
R′₂, R′₃=both H or either one is methyl and the other is H;
d′=an integer having a value of 0 to 2;
R′₄, R′₅=alkyl having 1 to 18 carbon atoms;
R′₆, =methyl, a mixture of organic residues obtained from the addition of an epoxy compound to an acid and having the following structures: 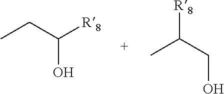
R′₈=H, alkyl, or methyl versatate radical;
L′=represents nil or divalent organic residue having the following structure: 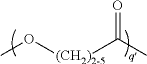
q′ is an integer having a value of 0 to 3;
z′=an integer or fraction having a value between 0 and 1;
x′, y′=a fraction having a value between 0 and 0.8, the sum of x+y is an integer of 0 or 1, and the sum of x′, y′ and z′ is 1;
u′, n′, m′, p′=each is an integer having a value, independently, of 0 or 1, and the sum of u′, n′, m′, and p′ is at least 2 and not more than 40; and
R′₇=monovalent alkyl radical having 1 to 18 carbon atoms.

5. The amino and hydroxy-functional compound according to claim 1, wherein the compound is in the form of a compound having a general structure of Formula III: 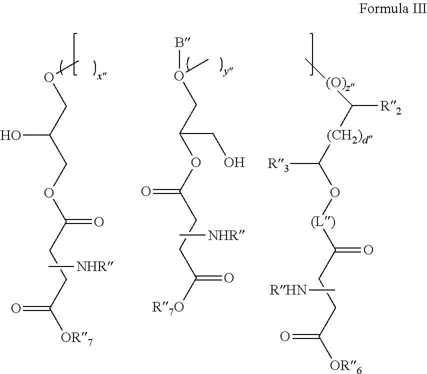
wherein: B″ represents the backbone of Formula III and B″ is an organic residue obtained from either a polyol after removing a hydroxyl groups and/or from an epoxy compound after removing oxirane rings where such a backbone is based on at least one of: hydrocarbon residue, a polyether residue, a polyurethane residue, a polycarbonate residue or any other backbone residue having a valency between 1 to 10, and
wherein:
R″=mono-valent alkyl, aryl and/or arylalkyl radical;
R″₁=H, methyl;
R″₂, R″₃=both H or either one is methyl and the other is H;
d″=an integer having a value of 0 to 2;
R″₄, R″₅=alkyl having 1 to 18 carbon atoms;
R″₆, =methyl, a mixture of organic residues obtained from the addition of an epoxy compound to an acid and having the following structures: 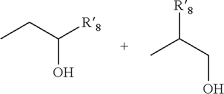
R′₈═H, alkyl, or methyl versatate radical;
L″=represents nil or divalent organic residue having the following structure: 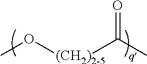
q′ is an integer having a value of 0 to 3;
z″=an integer or fraction having a value between 0 and 1;
x″, y″=a fraction having a value between 0 and 0.8, the sum of x+y is an integer of 0 or 1, and the sum of x″, y″ and z″ is 1; and
R″₇=monovalent alkyl radical having 1 to 18 carbon atoms.

6. The amino and hydroxy-functional compound according to claim 1, wherein the number average molecular weight of the amino and hydroxy-functional polymer is about 500 or higher and about 5,000 or lower.

7. The amino and hydroxy-functional compound according to claim 1, wherein the polydispersity of the amino and hydroxy-functional compound is about 4 or lower, and about 1.2 or higher.

8. The amino and hydroxy-functional compound according to claim 1, wherein the amino and hydroxy-functional compound has a hydroxyl value of about 40 or higher and of about 300 or lower.

9. The amino and hydroxy-functional compound according to claim 1, wherein the amino and hydroxy-functional polymer has an amine value of about 40 or higher and of about 300 or lower.

10. The amino and hydroxy-functional compound according to claim 1, wherein the amino and hydroxy-functional polymer has an average total functionality of about 1.8 or more and of about 10 or less.

11. Curable compositions comprising the amino and hydroxy-functional compound according to claim 1 and a polyisocyanate, wherein the amount of isocyanate is present in about 60% of the molar amount of the amino and alcohol groups, or more.

12. A process for preparing an amino and hydroxy-functional compound, wherein a hydroxyl functional acrylic polymer or other polyol is reacted with maleic acid anhydride producing newly generated free acid groups, and wherein the newly generated free acid groups are converted to esters by reaction with an epoxy compound and wherein at least one of a maleate and fumarate unsaturation is reacted with an aliphatic or aromatic primary monoamine to prepare a corresponding aspartate derivative.

13. A process for preparing an amino and hydroxy-functional compound, wherein an epoxy functional acrylic polymer or other epoxy compound is reacted with monoalkylmaleate half acid-ester to convert epoxy group into ester groups and wherein at least one of maleate and fumarate unsaturation is reacted with an aliphatic or aromatic primary monoamine to prepare a corresponding aspartate derivative.

14. A coating composition comprising the following components in parts by weight (pbw):
(a) amino and hydroxy-functional compound (1-80 pbw), wherein the amino and hydroxy-functional compound is not a polyester, and wherein the compound comprises an amine in the form of aspartic acid esters functionality, and wherein the amino and hydroxy-functional compound has
(i) a molecular weight (Mn) of at least about 500;
(ii) an acid value of about 5 or less;
(iii) a hydroxyl value of about 30 or more; and
(iv) an amine value of about 30 or more.
(b) polyisocyanate compound (1-65 pbw)
(c) other binder constituents (0-60 pbw)
(d) colorants (0-40 pbw)
(e) additives (0-10 pbw)
(f) tin catalyst (0-0.1 pbw)
(g) solvents (0-30 pbw)
wherein components (a)-(f) together are 100 pbw.

15. The composition according to claim 14, wherein component (c) is present in an amount between 1 and 60 pbw, and wherein component (c) comprises one or more of:
(i) hydroxy functional acrylic polymers, hydroxy functional polyester, hydroxy functional reactive diluent, hydroxy functional polyether, hydroxy functional polycarbonate or hydroxy functional polyurethane;
(ii) non-functional polymers or functional polymers with a functionality equivalent weight of about 5000 or higher; and
(iii) aspartate functional compounds other than compound (a).

16. The composition according to claim 14, wherein component (c) is present in an amount between 5 and 50 pbw.

17. The composition according to claim 14, wherein component (c) comprises a hydroxy functional acrylic polymer.

18. The composition according to claim 14, wherein component (c) comprises an aspartate functional compound other than component (a).

19. The composition according to claim 14, wherein component (g) is present in about 10 pbw relative to components (a) through (f) or less.
